(19) United States
(12) Patent Application Publication
(10) Pub. No.: US 2018/0221187 A1
(43) Pub. Date: Aug. 9, 2018

(54) ADJUSTABLE ORTHOTIC FOOT BRACE AND METHOD FOR ADJUSTING A FOOT BRACE

(71) Applicant: ORTHÈSES TURBOMED INC. / TURBOMED ORTHOTICS INC., Québec (CA)

(72) Inventor: Stéphane SAVARD, Québec (CA)

(21) Appl. No.: 15/750,691

(22) PCT Filed: Jun. 23, 2016
(86) PCT No.: PCT/CA2016/050733

§ 371 Date: Feb. 6, 2018

(30) Foreign Application Priority Data
Aug. 7, 2015 (CA) 2900058

(51) Int. Cl. A61F 5/01

(52) U.S. Cl. CPC A61F 5/0113

(57) ABSTRACT

The orthotic foot brace for a person wearing a footwear generally has: a lower leg holder securable to a lower leg of the person for use, a lower leg strut connected to the lower leg holder and extending downwardly towards the footwear during use; a foot strut structurally connected to the lower leg strut and having at least one side portion, each side portion extending forwardly along a respective side of the footwear and extending outwardly along a respective side of the footwear; an instep strut provided in the form of an extension of the foot strut along a long axis of the footwear, the instep strut having a distal portion securable to the footwear, and at least a proximal portion being slidably connected to the foot strut in a manner to allow adjusting the extension distance of the distal portion to a selected position.

### FIELD

The improvements generally relate to orthotics and, more particularly, to an orthosis for remediation of foot drop symptoms.

### BACKGROUND

Foot drop, drop foot, and foot dangle are terms which have been employed to describe ankle and toe dorsiflexor paresis (referred to hereinafter as foot drop for simplicity) resulting in the inability to raise the foot at the ankle, such that the foot inclines towards and scrapes the ground when walking. Dorsiflexion is the motion the ankle joint makes when the foot points upward. This motion needs to occur when the foot comes off the ground so that the toes do not drag.

Several braces have been developed for foot drop. An example of an orthotic foot brace developed for foot drop is published under international patent publication number WO 2011/097723. Such an orthotic foot brace is shown at 10 in FIG. 1. This brace, as opposed to other existing braces for foot drop, allows plantarflexion (i.e. the foot can point downward) and is designed to fit outwardly of a given size of footwear. Although satisfactory to a certain degree, there remains room for improvement.

### SUMMARY

The brace described in international patent publication number WO 2011/097723 is a commercial success through electronic commerce (e.g., via Internet-based platforms). However, a factor which currently limits its adoption is the fact that some orthotists (or other orthotic foot brace providers) find it inconvenient to keep an inventory of different sizes to fit with different sizes of footwear of potential buyers who would wish to try the orthotic foot brace prior to purchasing it. Indeed, the inventory typically requires an initial investment and storage area.

In accordance with one aspect, there is provided an orthotic foot brace which has a telescoping portion operable to adjust the orthotic foot brace to different sizes of footwear.

In accordance with one aspect, there is provided an orthotic foot brace for a person wearing a footwear comprising: a lower leg holder securable to a lower leg of the person for use, a lower leg strut connected to the lower leg holder and extending downwardly towards the footwear during use; a foot strut structurally connected to the lower leg strut and having at least one side portion, each side portion extending forwardly along a respective side of the footwear and extending outwardly along a respective side of the footwear during use; an instep strut provided in the form of an extension of the foot strut along a long axis of the footwear during use, the instep strut having a distal portion securable to the footwear during use, and at least a proximal portion being slidably connected to the foot strut in a manner to allow adjusting the extension distance of the distal portion to a selected position for use.

In accordance with another aspect, there is provided a method for adjusting an orthotic foot brace to be worn by a person, the orthotic foot strut having a lower leg holder, a lower leg strut connected to the lower leg holder and extending away from the lower leg holder; a foot strut structurally connected to the lower leg strut and extending away from the lower leg strut, the method comprising the steps of: securing the lower leg holder to a lower leg of the person; securing the foot strut adjacent the footwear of the person, the foot strut pointing towards an instep portion of the footwear; and sliding an instep strut towards the foot strut thereby adjusting the position of a distal portion of the instep strut for securing the distal portion to the footwear.

In accordance with another aspect, there is provided an orthotic foot brace for a person wearing a footwear comprising: a lower leg holder securable around a lower leg of the person; a lower leg strut secured to the lower leg holder and extending downwardly towards the footwear; a foot strut connected to the lower leg strut and having at least one of a median portion and a lateral portion juxtaposable to a respective side of the footwear and outwardly thereof, and a front portion securable to an instep portion of the footwear; and at least one brace retaining member connected to the foot strut, juxtaposable to the footwear and having at least a portion extending on the median side of the footwear and at least a portion extending on the lateral side of the footwear, outwardly thereof, and restraining at least one of a rearwardly and downwardly oriented pivotal movement of the brace characterized in that the front portion is slidable to allow adjusting an extension distance of the front portion to a selected position for use by the person.

In accordance with another aspect, there is provided an orthotic foot brace for a person wearing a footwear comprising: a frame including a lower leg holder securable to a lower leg of the person; a vertical portion secured to the lower leg holder and extending downwardly towards the footwear; a foot portion secured to the vertical portion, extending outwardly of the footwear and forwardly towards an instep portion of the footwear and securable to the footwear; and a brace retaining member connected to the foot portion, extending from a median side of the footwear to a lateral side of the footwear, and restraining at least one of a rearwardly and a downwardly oriented pivotal movement of the brace characterized in that the foot portion is slidable to allow adjusting an extension distance of the front portion to a selected position for use by the person.

Many further features and combinations thereof concerning the present improvements will appear to those skilled in the art following a reading of the instant disclosure.

### DESCRIPTION OF THE FIGURES

In the figures,

FIG. 1 is an oblique view of an example of a conventional orthotic foot brace, in accordance with the prior art;

FIG. 2 is an oblique view of an example of an adjustable orthotic foot brace, showing an instep strut disconnected from a foot strut of the adjustable orthotic foot brace, in accordance with an embodiment;

FIGS. 3A and 3B are partial side views of an adjustable orthotic foot brace having an instep strut adjusted in two different extended positions, in accordance with two embodiments;

FIG. 4 is a side elevation view of an adjustable orthotic foot brace during use, in accordance with an embodiment;

FIG. 5A shows a front view of an adjustable orthotic foot brace, with no instep strut connected to a foot strut of the adjustable orthotic foot brace, in accordance with an embodiment;

FIG. 5B shows an enlarged portion of the front view of FIG. 5A, showing a strut connector, in accordance with an embodiment;

FIGS. 6A and 6B are top plan views of two different instep struts having different lengths, in accordance with two embodiments;

FIGS. 7A and 7B are partial side views of an adjustable orthotic foot brace having a heel retaining strut adjusted in two different extended positions, in accordance with two embodiments; and

FIGS. 8A and 8B are side views of an adjustable orthotic foot brace having a lower leg holder adjusted in two different extended positions, in accordance with an embodiment.

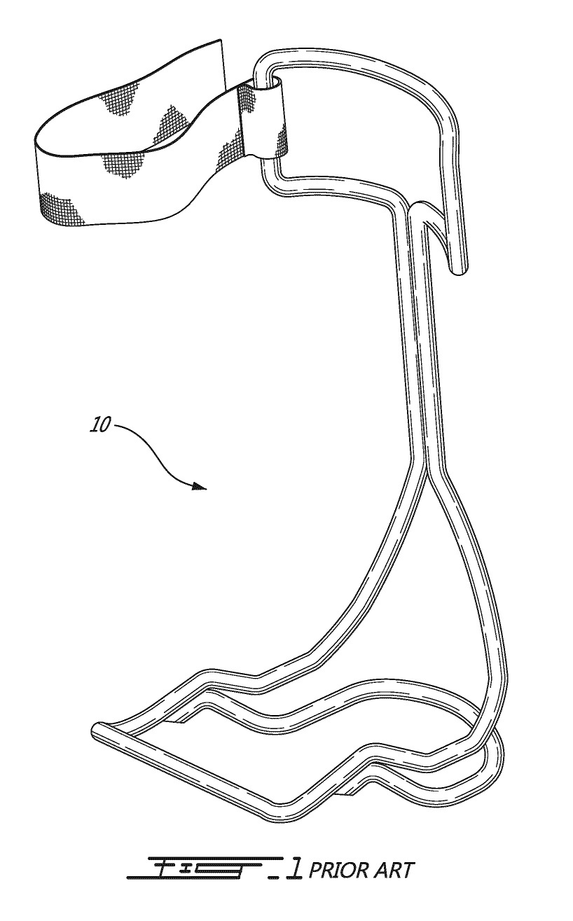

### DETAILED DESCRIPTION

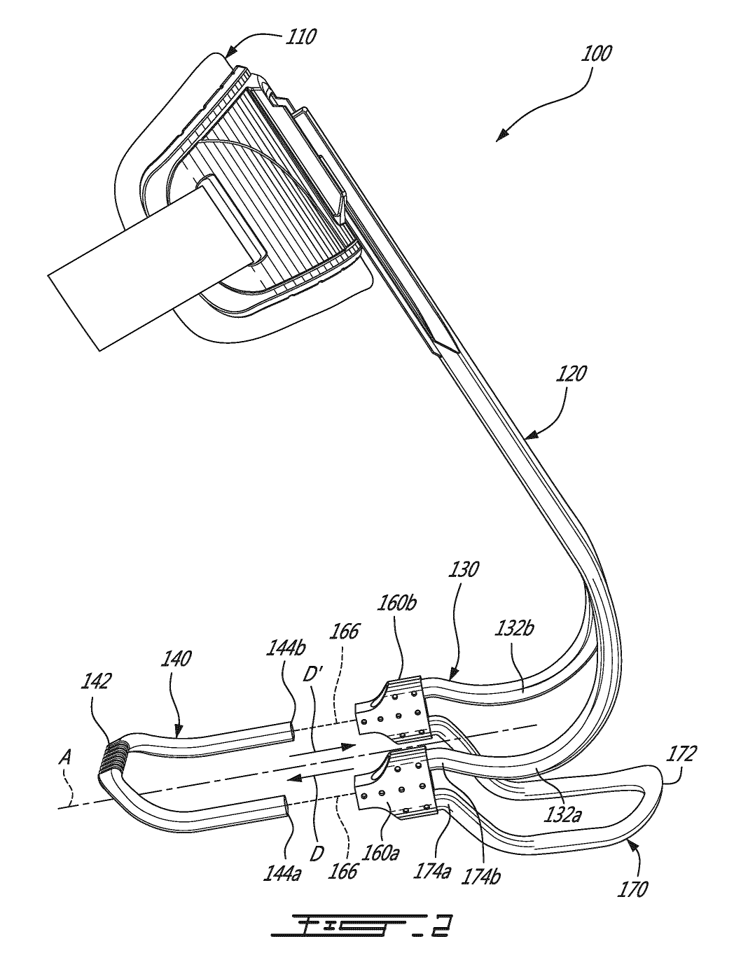

FIG. 2 shows an adjustable orthotic foot brace 100, in accordance with an embodiment. As depicted, the adjustable foot brace 100 has a lower leg holder 110, a lower leg strut 120, a foot strut 130, and an instep strut 140 adjustable along a long axis A of the footwear of the patient during use (see forward direction D and rearward direction D′). As it will be understood, the instep strut 140 is slidably mounted to the foot strut 130, in a manner that a distal end of the foot strut 130 can be moved to a selected position to adapt it to a corresponding size of the footwear of the patient.

The adjustable orthotic foot brace is hereinafter referred to simply as “adjustable foot brace 100” for ease of reading. Although the adjustable foot brace 100 can be used by a foot drop patient in her/his everyday physical activities and/or for special athletic events, for instance, the adjustable foot brace 100 can be particularly useful for orthotists and/or orthotic brace providers.

An orthotist can use the adjustable foot brace 100 in order to determine which non-adjustable orthotic foot brace size is best adapted for a given foot drop patient. More specifically, the orthotist can position the adjustable foot brace 100 to the footwear of the patient. The orthotist may then obtain measurements associated with one or more component of the non-adjustable orthotic foot brace. The measurements can be used in order to place an order, potentially to an orthotic foot brace manufacturer, of an orthotic foot brace 10 sized to the specific foot size of the foot drop patient. It was found that orthotists can prefer to purchase a smaller amount of adjustable foot braces 100 to allow patients to try the feeling of the brace prior to purchase, rather than holding an inventory of orthotic foot braces of all required different sizes for this purpose.

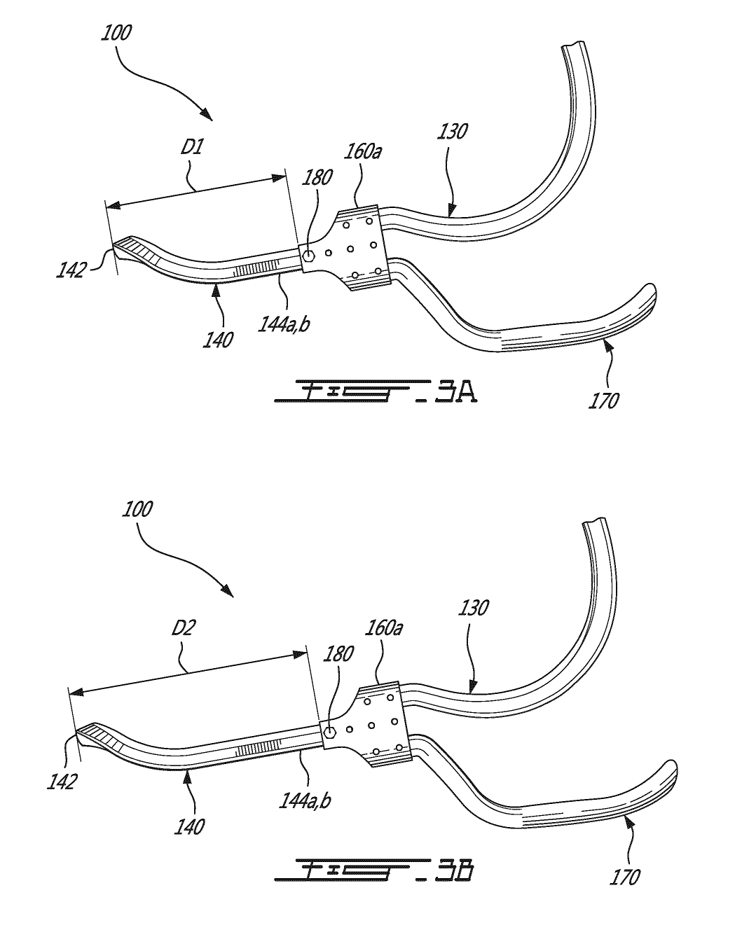

For instance, FIG. 3A shows the instep strut 140 when adjusted to extend to a first selected position defining a first extension distance D1 between the distal portion 142 and the foot strut 130 while FIG. 3B shows the instep strut 140 adjusted to extend to a second selected position defining a second extension distance D2, greater than the first extension distance D1. As will be understood, the adjustable foot brace 100 as adjusted in the embodiment shown in FIG. 3A can be best fitted for smaller footwear while the adjustable foot brace 100 as adjusted in the embodiment shown in FIG. 3B is best suited for footwear of greater dimensions.

As will be understood by the skilled reader, the instep strut 140 is lockable, into a specific position, to the strut connectors 160a,b using a fastener 180 such as a screw (e.g., a set screw) or any other suitable fastener.

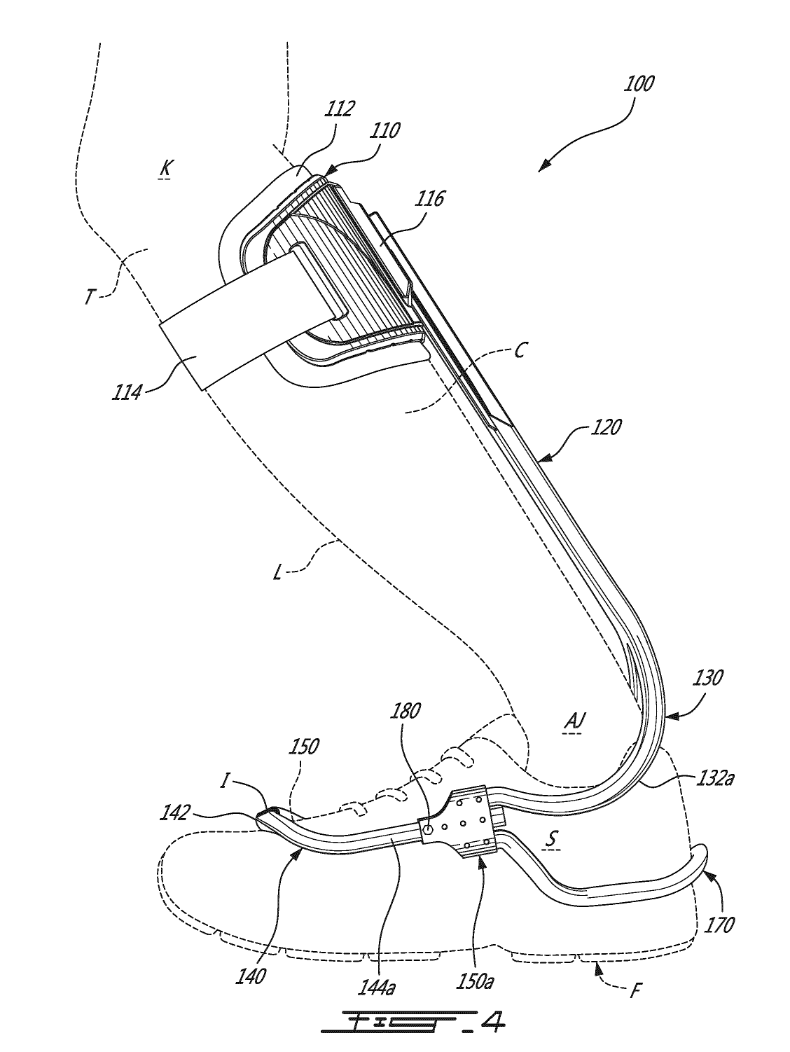

For ease of reading, the adjustable foot brace 100 is now described with reference to FIG. 4, which shows the adjustable foot brace 100 during use, i.e. when secured to the lower leg L of the patient and to the footwear F worn by the patient. As shown, the lower leg holder 110 is secured to the lower leg L of the patient and forms a first point of contact between the adjustable foot brace 100 and the patient. The lower leg holder 110 is meant to be secured anywhere below the knee K of the patient and along the lower leg L of the patient. In the embodiment shown, the lower leg holder 110 is provided in the form of a calf cuff 112 and of a leg attachment strap 114. The calf cuff 112 is abutted on an upper part of the calf C of the patient, below the knee K. In the embodiment shown, the leg attachment strap 114 extends toward the front of the tibia T of the patient and encircles the lower leg L of the patient, which secures an upper part of the adjustable foot brace 100 to the lower leg L of the patient. In an alternate embodiment, the lower leg holder has a different shape than the one shown in the figures.

The lower leg strut 120 is connected to the lower leg holder 110. In the embodiment shown in FIG. 4, the lower leg strut 120 is connected to a rear portion 116 of the lower leg holder 110, rearwardly of the calf C of the patient. The lower leg strut 120 extends away from the lower leg holder 110 and downwardly towards the footwear F of the patient. In the embodiment shown, the lower leg strut 120 is a substantially flat member. However, it is appreciated that it can be any other suitable shapes, such as a rod having a circular cross section, for instance.

As shown in FIG. 4, the foot strut 130 is structurally connected to the lower leg strut 120. In other words, the lower leg strut 120 and the foot strut 130 are provided in the form of a single piece of material. In another embodiment, the foot strut 130 and the lower leg strut 120 are two separate pieces of material fixedly joined to one another. The illustrated embodiment shows that the joint between lower leg strut 120 and the foot strut 130 is substantially curvilinear. However, such a joint can differ in shape in another possible embodiment. Means of suitably joining the foot strut 130 to the lower leg strut 120 can vary. The foot strut 130, as opposed to the lower leg strut 120, is a rod-like member having a substantially circular cross section. In another embodiment, the foot strut 130 has another suitable shape.

As best seen in FIG. 2, the foot strut 130 has two side portions 132a,b, each being extending forwardly towards an instep portion of the footwear along a respective side of the footwear during use. For instance, referring back to FIG. 4, the side portion 132a extends along the side S of the footwear F.

The instep strut 140 provides a second point of contact between the adjustable foot brace 100 and the patient. More specifically, the instep strut 140 is securable to the footwear F (e.g., securable to the instep portion I of the footwear F). The adjustable foot brace 100 is satisfactorily adjusted when a distal portion 142 of the instep strut 140 can be secured to the footwear F. Typically, the instep portion I of the footwear F has a lace clip 150 secured to the laces of the footwear F. Accordingly, the instep strut 140 acts as an extension of the foot strut 130 along the long axis A of the footwear F in order to provide the distal portion 142 to the lace clip 150 while proximal portions 144a,b can slide relative to the foot strut 130. When the distal portion 142 is properly secured to the lace clip 150, the orthotist can measure a selected position at which the instep strut 140 extends towards the instep portion I of the footwear F. In the embodiment shown, the proximal portion 144a of the instep strut 140 extends continuously to the proximal portion 144b via the distal portion 142. During use of the adjustable foot brace 100, the instep strut 140 extends from a side of the footwear F to another side of the footwear F such that the instep strut 140 has a substantially C-shape. However, the instep strut 140 can have any other shape.

In the embodiment shown, the adjustability of the instep strut 140 relative to the foot strut 130 is provided by the strut connectors 160a,b. As it will be understood, the strut connector 160a connects the side portion 132a of the foot strut 130 and the proximal portion 144a of the instep strut 140, and the strut connector 160b connects the side portion 132b of the foot strut 130 and the proximal portion 144b of the instep strut 140. Although shown as separate parts, the strut connectors 160a,b can be made integral to the foot strut 130. In such an embodiment, the strut connectors 160a,b, the foot strut 130 and the lower leg strut 120 are provided in the form of a frame made of a single piece of material.

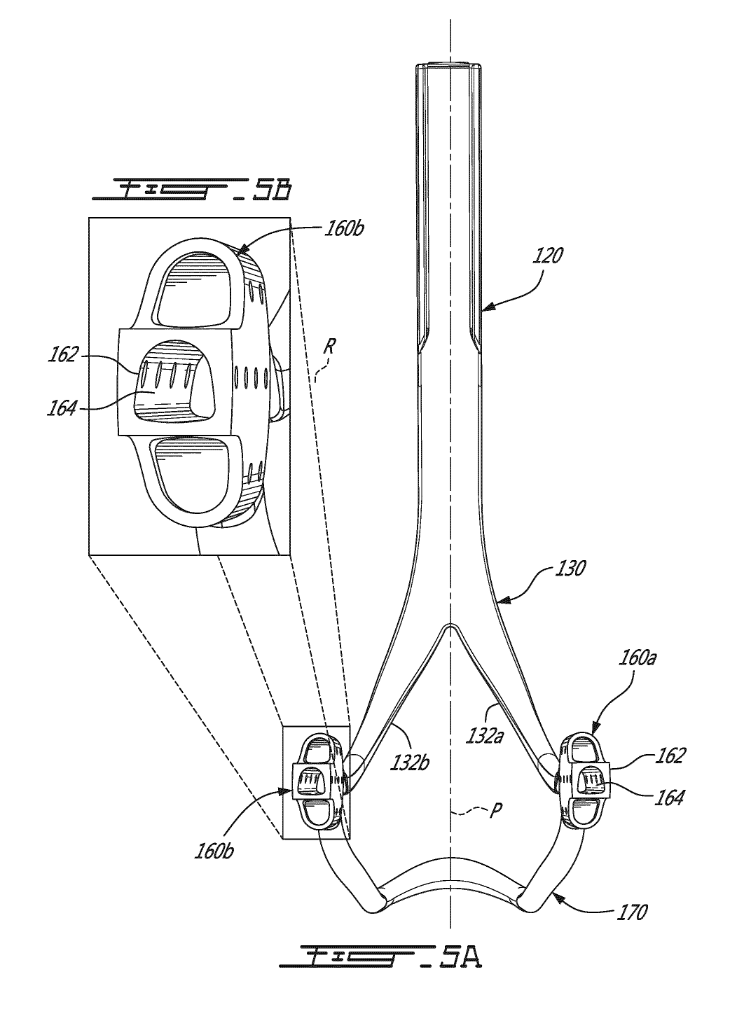

More specifically, and referring to FIGS. 5A-B, each of the strut connectors 160a,b has an inner wall 162 defining a bore 164 extending along an axis 166 (see reference numeral in FIG. 2) parallel to the long axis A of the footwear F during use. Accordingly, the proximal portions 144a,b of the instep strut 140 can each be provided into a corresponding one of the bores 164, thus allowing the instep strut 140 to slide relative to the foot strut 130. As it will be understood, the proximal portions 144a,b of the instep strut 140 have cross sections which snugly correspond to cross sections of the bores 164 in order to allow for a tight fitting. Furthermore, as best shown in FIG. 5B, the cross sections of the bores 164 are directional (asymmetry relative to a transverse axis R) such that the instep strut 140 can be slidably connected to the strut connectors 160a,b only in a satisfactory position. Indeed, without such directional cross sections, the instep strut 140 can be slidably connected to the strut connectors 160a,b when it is rotated by 180° about the long axis A of the footwear F during use, which would yield poor adjustment of the adjustable foot brace 100. As will be understood by the skilled reader, the instep strut 140 is lockable, into a specific position, to the strut connectors 160a,b using a fastener such as a screw (e.g., a set screw) or any other suitable fastener.

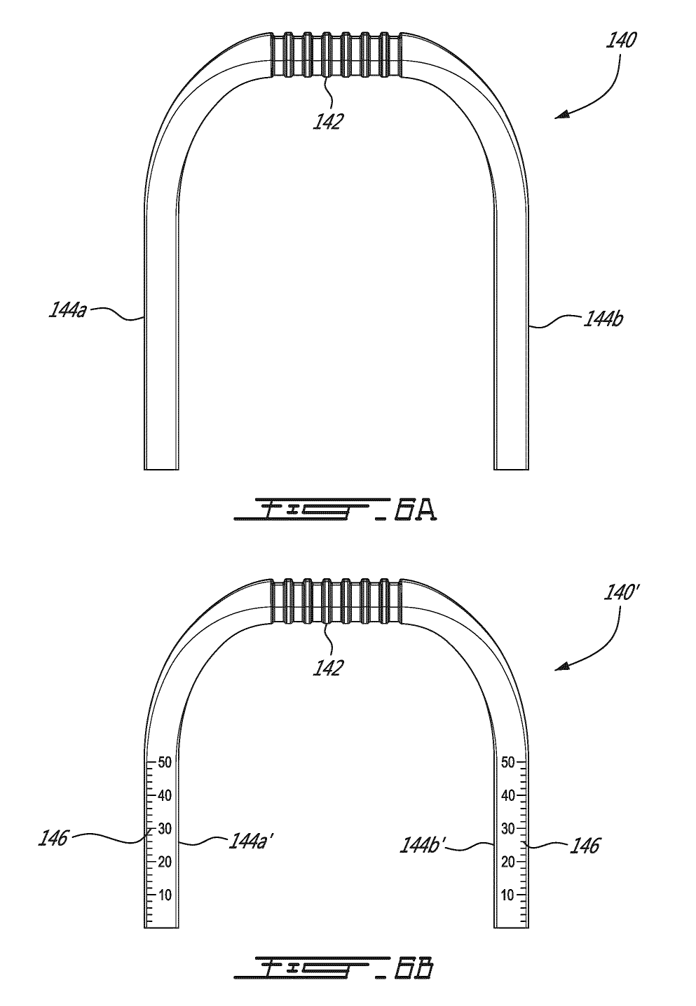

FIGS. 6A-B show two different examples of the instep strut at reference numerals 140 and 140′, in accordance with two embodiments. These embodiments show the other instep strut 140′ that can be used by the orthotist, for instance, when the footwear F of the patient is too short for the instep strut 140. Other step struts of different lengths can also be provided. Moreover, in the embodiment shown in FIG. 6B, the instep strut 140′ has a graduated scale 146 provided on the proximate portions 144a′,b′. In this embodiment, the graduated scale 146 is laser marked onto each of the proximate portions 144a′,b′ before being painted in an easily discernable color (e.g., white on a black substrate). In another embodiment, the graduated scale is provided in another satisfactory form (e.g., integrated to a sticker which is adapted to be applied).

In the embodiment shown, the adjustable foot brace 100 is symmetric relative to a sagittal plane P (perpendicular to the page in FIG. 5A). More specifically, the lower leg strut 120 can be symmetrical relative to the sagittal plane P of the lower leg L of the patient, and the foot strut 130, and associated side portions 132a,b, can also be symmetrical relative to the sagittal plane P of the lower leg L of the patient. In such an embodiment, such symmetry allows the adjustable foot brace 100 to be ambidextrous, i.e. it can be used for both the right leg and the left leg. In another embodiment, the adjustable foot brace is not symmetric relative to the sagittal plane P. For instance, an adjustable foot brace 100 can have a foot strut having a single side portion which extends along a respective side of the footwear during use. In this embodiment, the adjustable foot brace 100 can have a single strut connector connected to the single side portion of the foot strut and slidably connectable to a single proximate portion of an instep strut. Still in this embodiment, the instep strut has two ends, one at the proximate portion and one at an opposite, distal portion (securable to the footwear F).

It will be noted that the adjustable foot brace 100 has a heel retaining strut 170. The heel retaining strut 170 can be used to restrain a dorsiflexion (pivotal movement of the foot about ankle joint AJ, see FIG. 4) of the footwear F of the patient during use, as thoroughly described in international patent publication number WO 2011/097723. For the purpose of the adjustable foot brace 100, it is understood that the adjustable foot brace 100 can be used to suitably position the instep strut 140 relative to the foot strut 130 with or without the presence of the heel retaining strut 170. However, the heel retaining strut 170 can be provided to the adjustable foot brace 100 for convenience. Indeed, when an orthotist advises a patient on which adjustment of the instep strut 140 yields the best gait (more natural walk), the heel retaining strut 170 can be found useful. In some uses of the adjustable foot brace 100, however, the adjustable foot brace 100 is not meant to be used extensively by a patient, so the heel retaining strut 170 can be omitted.

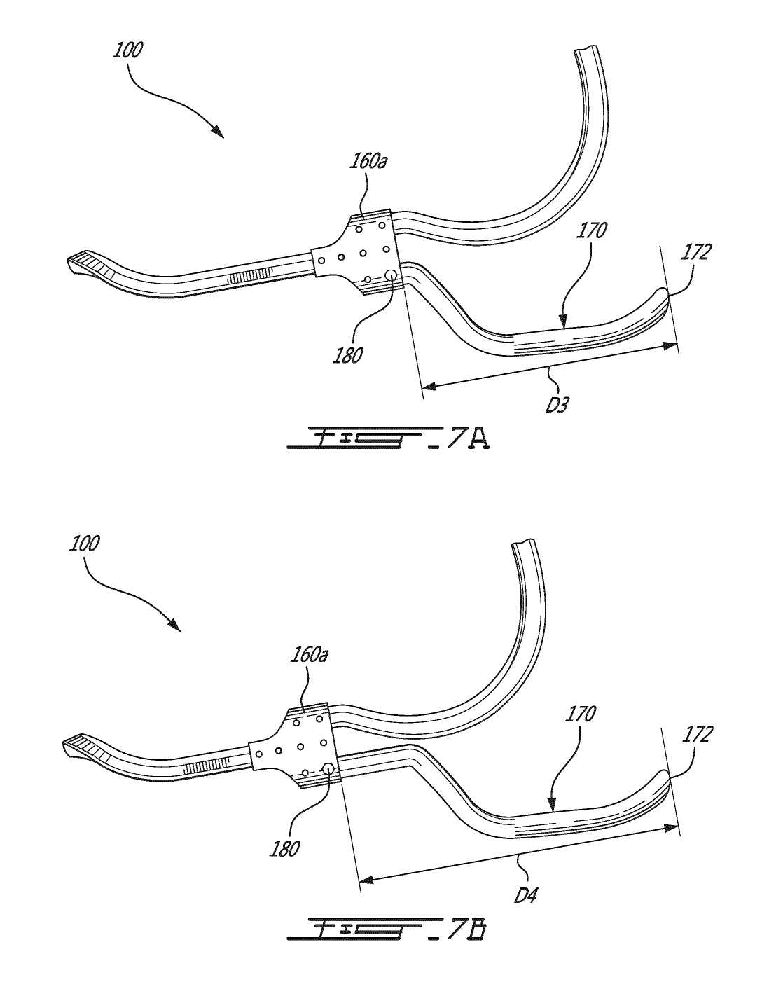

Turning now to FIGS. 7A-B, it will noted that the heel retaining strut 170 of the adjustable foot brace 100 can also be adjustable. More specifically, the heel retaining strut 170 can be slidably connectable to the strut connectors 160a,b. The heel retaining strut 170 has a distal portion 172 and two proximal portions 174a,b. In this embodiment, the relative terms distal and proximal are meant to be relative to the rearward direction D′ along the long axis A of the footwear during use. The proximal portions 174a,b of the heel retaining strut 170 slidably connect to the strut connectors 160a,b while the distal portion 172 of the heel retaining strut 170 points rearwardly. Accordingly, FIG. 7A shows the heel retaining strut 170 when adjusted to extend to a third selected position defining a third extension distance D3 between the strut connector 160a and a distal end 172 of the heel retaining strut 170 while FIG. 7B shows the heel retaining strut 170 adjusted to extend to fourth selected position defining a fourth extension distance D4, greater than the third extension distance D3. The fourth extension distance D4 has been exaggerated for simplicity and ease of understanding. However, although the adjustability of the heel retaining strut 170 is optional, it may be useful in some embodiments. In another embodiment, the heel retaining strut 170 may be lockable to the strut connectors 160a,b using a fastener 180 such as a screw (e.g., a set screw) or any other suitable fastener.

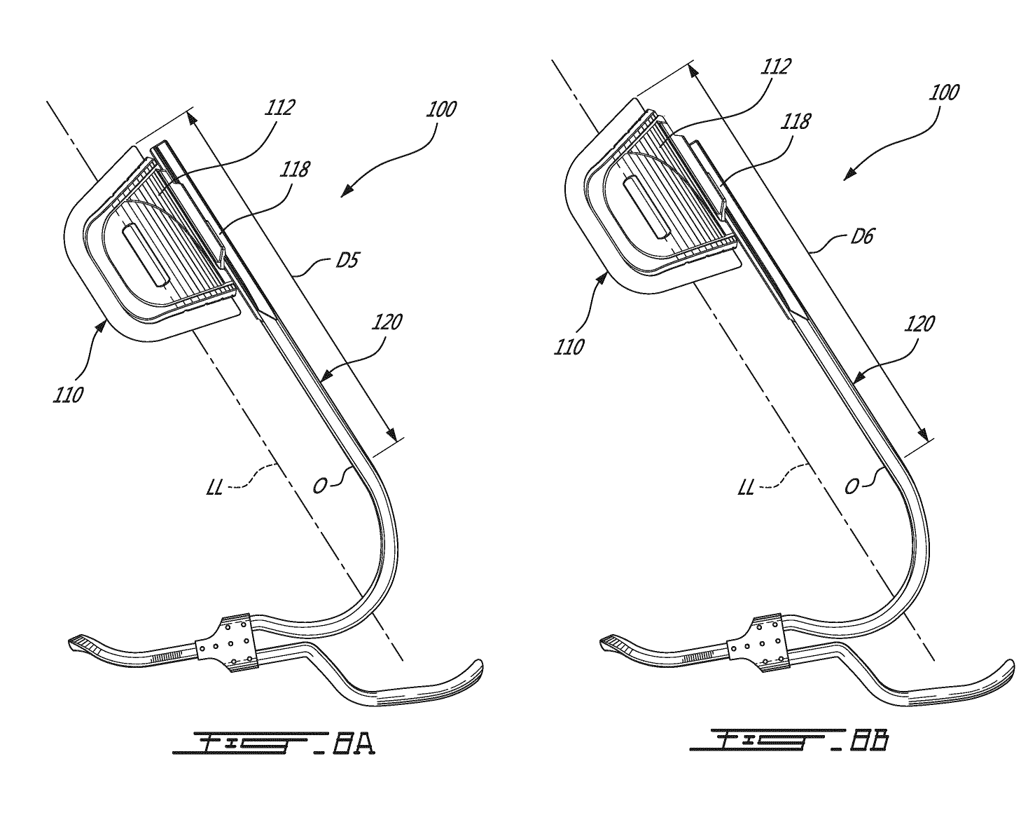

Furthermore, it is envisaged that the position of the lower leg holder 110 can be adjustable relative to the lower leg strut 120. Referring to FIGS. 8A-B respectively, the lower leg holder 110 can be positioned at fifth extension distance D5 and at a sixth extension D6, measured relative to a reference point O along the lower leg strut 120. In this embodiment, the rear portion of the calf cuff 112 is slidably connected to the lower leg strut 120 via a slidable dove joint 118. Indeed, in this embodiment, the slidable dove joint 118 has a longitudinal axis substantially parallel to a lower leg axis LL associated with the lower leg L. In this embodiment, the lower leg holder 110 is lockable to the lower leg strut 120 using a fastener 180 such as a screw (e.g., a set screw) or any other suitable fastener.

As briefly mentioned above, the orthotist can perform a method in order to adjust a foot brace and a method to obtain one or more foot brace measurement(s) associated with the adjustability of the adjustable foot brace 100, and especially of the instep strut 140, the heel retaining strut 170 and/or the lower leg holder 110, such as shown respectively in FIGS. 3A-B, in FIGS. 7A-B and in FIGS. 8A-B. This method can have a step of securing the lower leg holder 110 to the lower leg L of the patient. The method can have a step of securing the foot strut 130 adjacent the footwear F of the patient by making sure the foot strut 130 points towards the instep portion I of the footwear F of the patient. Once the footwear and the lower leg of the patient are properly secured within the adjustable foot brace 100, the method has a step of sliding the instep strut 140 towards the foot strut 130 thereby adjusting the distal portion 142 of the instep strut 140 such that the distal portion 142 can be secured to the lace clip 150 which is, in turn, secured to the footwear F. The method may have a step of obtaining the foot brace measurement by measuring an extension distance (e.g., D1, D2) between the foot strut 130 and the distal portion 142 of the instep strut 140. In this embodiment, once this measurement is properly done, the orthotist can measure another foot brace measurement (e.g., D3, D4) associated with the position of the heel retaining strut 170 that best fits the heel portion of the footwear F of the patient as well as measuring still another foot brace measurement (e.g., D5, D6) associated with the position of the lower leg holder 110 that best fits the patient.

As can be understood, the examples described above and illustrated are intended to be exemplary only. The expression instep is used herein broadly in a manner to encompass embodiments where the attachment to the shoe would be provided closer to the toes of the patient. The expression “use” (e.g., for use, during use) is used herein broadly to encompass use of the adjustable brace by a patient which is shopping for a foot brace (in addition to a user actually using the brace during a physical activity such as walking or running, for instance). The term patient is used to put the adjustable foot brace into the context of a foot drop affected person, but it is understood that any person can use the adjustable foot brace. The scope is indicated by the appended claims.

What is claimed is:

1. An orthotic foot brace for a person wearing a footwear comprising: a lower leg holder securable to a lower leg of the person for use, a lower leg strut connected to the lower leg holder and extending downwardly towards the footwear during use; a foot strut structurally connected to the lower leg strut and having at least one side portion, each side portion extending forwardly along a respective side of the footwear and extending outwardly along a respective side of the footwear during use; an instep strut provided in the form of an extension of the foot strut along a long axis of the footwear during use, the instep strut having a distal portion securable to the footwear during use, and at least a proximal portion being slidably connected to the foot strut in a manner to allow adjusting an extension distance of the distal portion to a selected position for use.

2. The orthotic foot brace of claim 1, further comprising at least one strut connector slidably connecting each one of the at least one proximal portion of the instep strut to the foot strut.

3. The orthotic foot brace of claim 2, wherein each strut connector is made integral to the foot strut.

4. The orthotic foot brace of claim 2, wherein each strut connector has an inner wall defining a bore, the bore having a longitudinal axis parallel to the long axis of the footwear, each proximal portion of the instep strut being slidable along the respective bore and lockable at the selected position.

5. The orthotic foot brace of claim 4, wherein the inner wall of the bore has a directional cross section snugly corresponding to a directional cross section of the proximal portion of the instep strut.

6. The orthotic foot brace of claim 1, further comprising a heel retaining strut connected to the foot strut and extending on each side of the footwear during use, the heel retaining strut restraining at least one of a rearwardly and downwardly oriented pivotal movement of the brace.

7. The orthotic foot brace of claim 6, wherein the heel retaining strut is provided in the form of an extension of the foot strut along a rearward direction of the long axis of the footwear during use, the heel retaining strut having a distal portion, and at least a proximal portion being slidably connected to the foot strut in a manner to allow adjusting the extension distance of the distal portion of the heel retaining strut to a second selected position for use.

8. The orthotic foot brace of claim 7, further comprising at least one strut connector slidably connecting one of the at least one proximal portion of the heel retaining strut to the foot strut.

9. The orthotic foot brace of claim 8, wherein each strut connector slidably connects one of the at least a proximal portion of the instep strut and one of the at least a proximal portion of the heel retaining strut.

10. The orthotic foot brace of claim 8, wherein each proximal portion of the heel retaining strut is lockable at the second selected position via the strut connector.

11. The orthotic brace of claim 1, wherein the lower leg holder is slidably connected to the lower leg strut in a manner to allow adjusting the lower leg holder along a lower leg axis at a third selected position during use.

12. The orthotic brace of claim 11, wherein the lower leg holder is lockable at the third selected position.

13. The orthotic foot brace of claim 1, wherein the instep strut has two proximal portions extending continuously from a side of the footwear to another side of the footwear via the distal portion of the instep strut.

14. The orthotic foot brace of claim 1, wherein the lower leg strut is symmetrical relative to a sagittal plane of the lower leg of the person.

15. The orthotic foot brace of claim 1, wherein the foot strut is symmetrical relative to a sagittal plane of the lower leg of the person.

16. The orthotic foot brace of claim 1, wherein the proximal portion has a graduated scale therealong to allow measurement of the selected position.

17. The orthotic foot brace of claim 1, further comprising more than one instep strut, each of the instep struts having at least one proximal portion of different lengths along the long axis of the footwear during use.

18. A method for adjusting an orthotic foot brace to be worn by a person, the orthotic foot strut having a lower leg holder, a lower leg strut connected to the lower leg holder and extending away from the lower leg holder; a foot strut structurally connected to the lower leg strut and extending away from the lower leg strut, the method comprising the steps of:
securing the lower leg holder to a lower leg of the person;
securing the foot strut adjacent the footwear of the person, the foot strut pointing towards an instep portion of the footwear; and
sliding an instep strut towards the foot strut thereby adjusting the position of a distal portion of the instep strut for securing the distal portion to the footwear.

19. The method of claim 18, further comprising obtaining a foot brace measurement by measuring an extension distance between the foot strut and the distal portion of the instep strut.

20. The method of claim 19, wherein said securing the lower leg holder further comprises adjusting the lower leg holder relative to the lower leg strut relative to the lower leg of the person during use, and obtaining another foot brace measurement between the lower leg holder and a tip of the lower leg strut.

21. The method of claim 19, further comprising transmitting the measurement to an orthotic foot brace provider providing a customized orthotic foot brace based on the measured extension distance.

22. An orthotic foot brace for a person wearing a footwear comprising: a frame including a lower leg holder securable to a lower leg of the person; a vertical portion secured to the lower leg holder and extending downwardly towards the footwear; a foot portion secured to the vertical portion, extending outwardly of the footwear and forwardly towards an instep portion of the footwear and securable to the footwear; and a brace retaining member connected to the foot portion, extending from a median side of the footwear to a lateral side of the footwear, and restraining at least one of a rearwardly and a downwardly oriented pivotal movement of the brace CHARACTERIZED IN THAT the foot portion is slidable to allow adjusting an extension distance of the front portion to a selected position for use by the person.
